(19) United States
(12) Patent Application Publication
(10) Pub. No.: US 2012/0232457 A1
(43) Pub. Date: Sep. 13, 2012

(54) Systems and Methods for Homeostatically Treating Organ Disease Using Local Delivery of Therapeutic Agents

(75) Inventor: Krishna Kandarpa, Southborough, MA (US)

(73) Assignee: DELCATH SYSTEMS, INC., New York, NY (US)

(21) Appl. No.: 13/508,729

(22) PCT Filed: Nov. 9, 2009
(86) PCT No.: PCT/US09/63744

§ 371 Date: May 8, 2012

(51) Int. Cl. A61M 1/34; A61M 1/00

(52) U.S. Cl. 604/6.09; 604/8; 604/5.01

(57) ABSTRACT

The instant invention encompasses a catheter, comprising: a first expandable occlusion device and a second expandable occlusion device, expandable beyond a wall of the catheter, the first occlusion device and the second occlusion device spaced along the catheter for generating an occluded segment of the blood vessel between the first occlusion device and the second occlusion device when the first occlusion device and the second occlusion device are expanded, a lumen or catheter for removing uncontaminated blood from a location up-stream of a first expandable occlusion device; a lumen or catheter for reintroducing the uncontaminated blood into a subject downstream from the second expandable occlusion device; apparati encompassing the above technology; and methods of use incorporating same.

### BACKGROUND

Current acceptable medical practice for treating diseases of body organs often involves surgical removal of the afflicted areas or surgical removal of the entire organ. For example, treatment of diseases and malignancies of the pancreas by surgical removal is particularly troublesome as the surviving patient has a limited life span. The pancreas is located behind the stomach and comprises two portions: one portion secretes digestive juices which pass into the duodenum; the other portion secretes insulin which passes into the bloodstream. The pancreas can become afflicted with two major types of tumors: ductal adenocarcinoma and endocrine tumors that can be either non-functioning tumors or functioning tumors. Non-functioning tumors can result in obstruction of the biliary tract or the duodenum, bleeding into the GI tract or be evidenced as abdominal masses. Functioning tumors can cause severe symptoms such as hypoglycemia, Zolinger-Elison syndrome, hypokalemia, carcinoid syndrome, and the like. When ductal adenocarcinoma is present, current treatment methods involve surgical removal of the affected areas it the cancer has not spread. Less than 2% of the patients undergoing this procedure survive for more than five years. When endocrine tumors are present, it is typical to surgically remove both the pancreas and the duodenum. In these instances, about 10% of the patients survive for five years.

The largest internal organ, the liver performs over 100 separate bodily functions; and its sheer complexity makes it susceptible to almost as many different diseases. Liver disease is a broad term describing any number of diseases affecting the liver including, but not limited to, hepatitis, cirrhosis, haemochromatosis, cancer of the liver (primary hepatocellular carcinoma or cholangiocarcinoma and metastatic cancers, usually from other parts of the gastrointestinal tract), Wilson's disease, primary sclerosing cholangitis, primary biliary cirrhosis, budd-chiari syndrome, Gilbert's syndrome, glycogen storage disease type II, biliary atresia, alpha-1 antitrypsin deficiency, alagille syndrome, and progressive familial intrahepatic cholestasis. Some organ diseases (such as those of the pancreas and/or liver) have also been treated in situ with toxic agents such as chemotherapeutic agents and other therapeutic biological agents that are toxic moieties obtained from organic sources. However, it has been found that these agents cannot generally be introduced into the main blood circulation of the body in sufficient strength and/or quantity to achieve desired therapeutic responses in the affected organs as their negative toxic effects on other organs and tissues of the body off-set their potential positive therapeutic effect in the afflicted organ.

### SUMMARY OF THE INVENTION

In one embodiment, the instant invention encompasses a catheter, comprising: a first expandable occlusion device and a second expandable occlusion device, expandable beyond a wall of the catheter, the first occlusion device and the second occlusion device spaced along the catheter for generating an occluded segment of the blood vessel between the first occlusion device and the second occlusion device when the first occlusion device and the second occlusion device are expanded, a lumen or catheter for removing uncontaminated blood from a location upstream of a first expandable occlusion device; and a lumen or catheter for reintroducing the uncontaminated blood into a subject downstream from the second expandable occlusion device.

In another embodiment, the instant invention encompasses a catheter in which the device for removing is sized and dimensioned for placement in the renal vein.

In another embodiment, the instant invention encompasses a catheter in which the extracorporeal circuit includes a filtration device for removing at least a portion of circulating hormones and/or other vasoactive agents present in the uncontaminated blood

In another embodiment, the instant invention encompasses an apparatus, part of which is positionable in a blood vessel of a body having a blood flow therethrough, the catheter having a first expandable occlusion device and a second expandable occlusion device, expandable beyond a wall of the catheter, the first occlusion device and the second occlusion device spaced along the catheter for generating an occluded segment of the blood vessel between the first occlusion device and the second occlusion device when the first occlusion device and the second occlusion device are expanded, the improvement in the catheter comprising: a first port in the wall of the catheter, the first port positioned upstream, in the direction of the blood flow from the first occlusion device; a second port in the wall of the catheter, the second port positioned downstream, in the direction of the blood flow from the second occlusion device; a lumen within the catheter and having a first end and a second end, the first end connected to the first port and the second end connected to the second port for defining a bypass for blood in the blood flow for shunting the occluded segment of the blood vessels spaced and positioned for passing a portion of the blood from upstream in the direction of blood flow from the occlusion to downstream in the direction of blood from the occlusion; and an extracorporeal circuit comprising a lumen for removing uncontaminated blood from a location upstream of a first expandable occlusion device; a device for extracorporeally pumping the removed uncontaminated blood back into a subject; and a lumen or catheter for reintroducing the uncontaminated blood into a subject downstream from the second expandable occlusion device.

In another embodiment, the instant invention encompasses a catheter comprising: a first lumen utilized to inflate/position a first occlusion device; a second lumen having perforations that may be utilized to convey blood draining into the occluded space between two inflated/positioned occluding device to an extracorporeal variable speed pump device and filtering device; a third lumen dimensioned and sized by providing a passage for blood to flow through from upstream to downstream of an occluded segment of the blood vessel; a fourth lumen that is connectable to an extracorporeal circuit for uncontaminated blood; and a fifth lumen that may be utilized to inflate/position a second occlusion device.

In another embodiment, the instant invention encompasses an apparatus comprising: a first lumen utilized to inflate/position a first occlusion device; a second lumen having perforations that may be utilized to convey blood draining into the occluded space between two inflated/positioned occluding device to an extracorporeal variable speed pump device and filtering device; a third lumen dimensioned and sized by providing a passage for blood to flow through from upstream to downstream of an occluded segment of the blood vessel; a fourth lumen connected to an extracorporeal circuit for uncontaminated blood, wherein the extracorporeal circuit for uncontaminated blood comprises: a lumen for removing uncontaminated blood from a location upstream of a first expandable occlusion device; a device for extracorporeally pumping the removed uncontaminated blood back into a subject; and a lumen or catheter for reintroducing the uncontaminated blood into a subject; and a fifth lumen that may be utilized to inflate/position a second occlusion device.

In another embodiment, the instant invention encompasses an apparatus comprising: a first lumen utilized to inflate/position a first occlusion device; a second lumen having perforations that may be utilized to convey blood draining into the occluded space between two inflated/positioned occluding device to an extracorporeal variable speed pump device and filtering device device; a third lumen connected to an extracorporeal circuit for uncontaminated blood, wherein the extracorporeal circuit for uncontaminated blood comprises: a lumen for removing uncontaminated blood from a location upstream of a first expandable occlusion device; and a lumen for extracorporeally pumping the removed uncontaminated blood back into a subject; a lumen or catheter for reintroducing the uncontaminated blood into a subject; and a fourth lumen that may be utilized to inflate/position a second occlusion device.

### BRIEF DESCRIPTION OF THE DRAWINGS

For a fuller understanding of the disclosure, reference is made to the following description taken in conjunction with the accompanying drawing(s) in which:

FIG. 1 shows a diagrammatic and schematic view of an embodiment of some of the main components of a system of the present invention in relationship to a body.

FIG. 2 shows a partial cross-sectional side view of an embodiment of a double occlusion device catheter useful in the process of the invention.

FIG. 3 shows a cross-sectional end view of the shaft of the double occlusion device catheter of FIG. 2.

FIG. 4 shows a cutaway cross-sectional side view of the interior of a double occlusion device catheter encompassed by the invention.

FIG. 5 shows a partial cross-sectional side view of an embodiment of a cartridge-type blood filtration device for use with the system of the present invention. The cartridge includes an adsorbent material.

FIG. 6 shows a partial cross-sectional side view of an embodiment of a hollow-fiber blood filtration device for use with the system of the present invention.

FIG. 7 shows a partial cross-sectional side view of an embodiment of a double occlusion device catheter with active bypass segment as described herein.

FIG. 8 shows a partial cross-sectional side view of an alternative embodiment of a double occlusion device catheter with active bypass segment as described herein.

FIG. 9 shows a partial cross-sectional side view of an alternative embodiment of a double occlusion device catheter with active bypass segment as described herein.

FIG. 10 shows a diagrammatic and schematic view of an embodiment of some of the main components of a system of the present invention in relationship to a body.

FIG. 11 shows a diagrammatic and schematic view of an alternative embodiment of some of the main components of a system of the present invention in relationship to a body.

### DETAILED DESCRIPTION

In one embodiment, the present invention relates to a system and method for the in situ treatment of organ disease using local delivery of therapeutic agents. A method for perfusing a high concentration of a therapeutic agent through a diseased organ of a body includes: perfusing a high concentration of a therapeutic agent therapeutic agent through the diseased organ, wherein the perfusion does not contaminate a general circulation of the body; removing contaminated blood from the organ, wherein the contaminated blood (whose concentration may be controlled by choice from zero to hundred percent by the filters) includes the therapeutic agent with effluent blood; transporting the contaminated blood to a blood filtration device; treating the contaminated blood in the blood filtration device to remove the contamination resulting in treated blood; returning the treated blood to the body. In another embodiment, the present invention further relates to providing a device for both passing upstream uncontaminated blood through the circulatory system past the occluded section of a particular blood vessel to a location in the blood vessel downstream to the occluded section; and mitigating effects on blood pressure that may result from the temporary occlusion of a blood vessel (in one embodiment, the inferior vena cava). The process may substantially prevent toxic levels of the therapeutic agent from entering the body's general circulation while delivering lethal doses of them to the diseased organ, and further provides for maintaining relative homeostasis of blood pressure despite the temporary occlusion of a major blood vessel.

As used herein, the term “therapeutic agent” refers to an agent used to treat a diseased organ. For example, in treating cancers an antineoplastic agent, such as a chemotherapeutic agent, may be used. For example, for treating hepatitis an interferon, such as interferon-α-2b or interferon-α-2a may be used. Examples of therapeutic agents for use with the systems and methods of the present invention include, but are not limited to, Abarelix, Aldesleukin, Aldesleukin, Alemtuzumab, Alitretinoin, Allopurinol, Altretamine, Amifostine, Amifostine, Amifostine, Anakinra, Anastrozole, Arsenic trioxide, Asparaginase, Asparaginase, Azacitidine, Azacitidine, Bevacuzimab, Bexarotene capsules, Bexarotene gel, Bleomycin, Bortezombi, Bortezomib, Busulfan intravenous, Busulfan oral, Calusterone, Capecitabine, Carboplatin, Carmustine, Celecoxib, Cetuximab, Chlorambucil, Cisplatin, Cladribine, Clofarabine, Cyclophosphamide, Cytarabine, Cytarabine liposomal, Dacarbazine, Dactinomycin, Actinomycin D, Dalteparin sodium, Darbepoetin alfa, Dasatinib, Daunorubicin liposomal, Daunorubicin, Daunomycin, Decitabine, Denileukin, Denileukin Diftitox, Dexrazoxane, Docetaxel, Doxorubicin, Doxorubicin liposomal, Dromostanolone Propionate, Eculizumab, Elliott's B Solution, Epirubicin, Epirubicin HCL, Epoetin alfa, Erlotinib, Estramustine, Etoposide phosphate, Etoposide, VP-16, Exemestane, Fentanyl citrate, Filgrastim, Floxuridine (intraarterial), Fludarabine, Fluorouracil, 5-FU, Fulvestrant, Gefitinib, Gemcitabine, Gemcitabine, Gemcitabine HCL, Gemicitabine, Gemtuzumab Ozogamicin, Goserelin acetate, Histrelin acetate, Hydroxyurea, Ibritumomab tiuxetan, Idarubicin, Ifosfamide, Imatinib mesylate, Interferon, Interferon (pegylated), Interferon alfa-2a, Interferon alfa-2b, Irinotecan, lapatinib ditosylate, lenalidomide, letrozole, leucovorin, Leuprolide Acetate, Levamisole, lomustine, CCNU, Meclorethamine, Nitrogen mustard, Megestrol acetate, Melphalan, Melphalan L-PAM, Mercaptopurine, 6-MP, Mesna, Methotrexate, Methoxsalen, Mitomycin C, Mitotane, Mitoxantrone, Nandrolone phenpropionate, Nelarabine, Nofetumomab, Oprelvekin, Oxaliplatin, Paclitaxel, Paclitaxel protein-bound particles, Palifermin, Pamidronate, Panitumumab, Pegademase, Pegaspargase, Pegfilgrastim, Peginterferon alfa-2b, Pemetrexed disodium, Pentostatin, Pipobroman, Plicamycin, Mithramycin, Porfimer sodium, Procarbazine, Quinacrine, Rasburicase, Rituximab, Sargramostim, Sorafenib, Streptozocin, Sunitinib, Sunitinib maleate, Talc, Tamoxifen, Temozolomide, Teniposide, VM-26, Testolactone, Thalidomide, Thioguanine, 6-TG, Thiotepa, Topotecan, Topotecan HCL, Toremifene, Tositumomab, Tositumomab/I-131 Tositumomab, Trastuzumab, Tretinoin, ATRA, Uracil Mustard, Valrubicin, Vinblastine, Vincristine, Vinorelbine, Vorinostat, Zoledronate, and Zoledronic acid.

As used herein, the term “occlusion device” denotes any of a variety of structures which may be reversibly inflated/positioned so as to occlude the blood vessel of a subject. Such structures include, but are not limited to, balloons; inflatable cuffs or sleeves; umbrella-shaped structures; fan-shaped structures; and all manners of fabric-covered wires or coils.

In one example, the method of the invention may avoid the use of surgery to isolate the flow of contaminated blood and returns the same blood but in a more purified condition to a patient; and further provides for techniques to assist in maintaining homeostasis of the patient's blood pressure during the time when a particular blood vessel is temporarily occluded. As a result, the method of the invention may be used for extended periods of time. The method of the invention is applicable to the treatment of primary and metastatic tumors, including all forms and sizes of tumor, as well as other diseases of an organ of a human body. In an embodiment, the organ is a liver. In an embodiment, the organ is a pancreas.

In an embodiment, the invention is directed to the treatment of tumors in the liver by the use of one or more antineoplastic agents, such as chemotherapeutic agents and/or biologicals, and the purification of venous blood from the liver to avoid systemic circulation of the agent(s). In an embodiment, the invention is directed to the treatment of hepatitis by the use of one or more therapeutic agents, such as interferon, and the purification of venous blood from the liver to avoid systemic circulation of the agent(s). In an embodiment, the invention is directed to the treatment of metastatic and primary cancers and tumors, including but not limited to, melanomas, adenocarcinomas, neuroendocrine tumors and hepatocellular carcinoma.

For treating a diseased liver, these treatment modalities may involve the use of double occlusion device catheters that are suitable for insertion in the inferior vena cava to isolate venous outflow from the liver and permit the removal of blood contaminated with therapeutic agent from the body. The contaminated blood captured by the double occlusion device catheter is fed through tubing to a blood purification device, possibly via the aid of a pump. The method will be successful even if the therapeutic agent is not completely removed from the blood. In one embodiment the amount of therapeutic agent in the body is kept below toxicity levels. One hundred percent removal of any drug is seldom possible and generally not practical.

In addition, these treatment modalities may involve the use of one or more accommodations so as to substantially maintain the normal flow of return blood in the occluded vessel and so as to facilitate homeostatis and management of blood pressure variations occasioned by the use of an occluding catheter in a major blood vessel.

In one embodiment, an occlusion bypass lumen is provided.

For treatment of tumors in the liver, because primary hepatocellular and metastatic hepatic tumors derive their blood supply from the hepatic artery, the tumor will be perfused by high concentrations of, for example, a chemotherapeutic agent. Because a normal liver receives three-fourths of its blood supply from the portal vein the agent will be diluted by a factor of about three before it reaches normal, uninvolved liver cells, thereby protecting them against hepatotoxicity.

The method of the invention involves the percutaneous placement of unique double occlusion device catheter designs. Double occlusion device catheter designs useful in practicing the invention are disclosed in, but not limited to, U.S. Pat. No. 5,069,662; U.S. Pat. No. 5,411,479; U.S. Pat. No. 5,817,046; U.S. Pat. No. 5,897,566; U.S. Pat. No. 5,919,163; U.S. Pat. No. 6,186,146 (now Abandoned) and U.S. Pat. No. 7,022,097, the disclosures of which are hereby incorporated herein by reference. One function of the double occlusion device catheter is to isolate the flow of blood from the veins carrying the effluent blood from the diseased liver. Venous isolation precludes systemic perfusion of the contaminated blood. Thus the tip of the double occlusion device catheter is to be placed in the body so that the venous effluent from the diseased liver being treated is prevented from flowing to the heart. The space between the two-occlusion devices is predetermined to ensure removing the full quantity of contaminated blood from the treated diseased liver. The space between the occlusion device is large enough that the occlusion device central in position can be located in a position in the most central draining vein to block contaminated venous blood flow to the heart and the occlusion device peripheral in position can be located peripheral in the most central draining vein to block the flow of uncontaminated blood to the contaminated venous blood flow. Veins from organs not under treatment can enter the segment between occlusion devices without detrimental effect as long as the blood filtration device can accommodate the additional volume. The venous anatomy of the diseased liver under treatment or of adjacent organs can be altered where necessary by obstruction using angiographic embolisation or ablation techniques and materials, including detachable occlusion devices or stainless steel coils.

The lumen of the catheter between the occlusion devices is openly connected, or can be made openly connected, to the surrounding vein. In addition, the same lumen of the catheter is also openly connected, or can be made openly connected, to the blood filtration device, thereby providing free flow of the contaminated blood from the veins to the blood filtration device. Thus the catheter has a main lumen to act as a conduit for the contaminated blood flow from the venous effluent(s) to the blood filtration device.

The size of the main lumen is determined by the material of which it is made, the volume of blood to be transported through it and the diameter of the vein in which it will be located. The main lumen may be an open annulus or semi-annulus located within the peripheral occlusion device that is openly connected to the extracorporeal circuit. In this type of catheter, a central rod or rodlike axis is provided for support for the occlusion device.

The catheter may also have supplemental lumina. The supplemental lumina are smaller in size, i.e., in diameter or cross-sectional area, than the main lumen. They may serve any of a number of ancillary functions in the process. For example, in one design, a supplemental lumen courses through the full length of the catheter for the purpose of accommodating a guidewire that is desirable for percutaneous insertion of the catheter. Each occlusion device may be provided with a supplemental lumen to be used for its inflation/positioning, or one supplemental lumen may be used for supplying fluid for the inflation/positioning of both occlusion devices. An additional supplemental lumen may be provided for connection to a pressure monitor to continuously measure the pressure of the venous effluent. This lumen can also be used to inject contrast medium, if provided with a connector that can accommodate an injection device. In some designs, the main lumen may be used for one or more of the above functions. This multifunctionality can serve to reduce the cost in making the catheter and simplify the apparatus. The main and/or supplemental lumina may be made from separate tubing threaded into the catheter or from channels molded into the structure of the catheter. Another supplemental lumen can be used to return detoxified blood to the general circulation and avoid puncture of another vein.

The wall(s) of the segment of the catheter between the occlusion devices are provided with fenestrations to allow entry of venous blood into the main lumen. The number, shape and size of the fenestrations may vary according to the size of the catheter, the rate and volume of blood they must transmit, and the materials of construction of the catheter. The shape and size of the fenestrations should take into consideration turbulence effects as the blood courses though the fenestrations and into the main lumen. Fenestrations that are too small can elevate hepatic sinusoidal pressure and fenestrations that are too large may weaken the catheter walls and compromise the integrity of the catheter.

One practical double occlusion device catheter design would have one large central lumen, 2 smaller lumina and 2 inflatable/positionable occlusion devices that are separated by about 9 to 10 cm. in the length of catheter that contains perforations. The catheter is designed to be positioned (under fluoroscopic guidance) in the inferior vena cava (IVC) such that the central occlusion device, when inflated/positioned, occludes the IVC just above the hepatic veins. The peripheral occlusion device, when inflated/positioned, occludes the IVC just below the hepatic veins, thus isolating hepatic venous blood from the systemic circulation. Perforations in the catheter between the two-inflated/positioned occlusion device convey blood through the large central catheter lumen to a variable speed pump and filtering device. An inferior vena cavagram through the main lumen can be used to document complete obstruction of the inferior vena cava proximal and distal to the hepatic veins. The effectiveness of passage of blood from the liver through the blood filtration device can be monitored by pressure measurement in the central catheter lumen. A variable speed pump may be adjusted to maintain normal hepatic vein pressure and flow. The detoxifying devices controllably reduce the agent in the blood to selected nontoxic levels before the blood is returned to the systemic circulation.

In another design an independent return lumen courses through the main lumen. One end to the return lumen is connected to the outlet of the blood filtration device and the other end openly outlets into a vein at a location superior to the diaphragm. When the double occlusion device catheter is located in the IVC, this return lumen extends beyond the end of the main catheter to the right atrium. In this construction, the return lumen consists of a separate piece of tubing threaded inside the main lumen and through the end hole of the catheter. The return lumen is large enough to carry the full volume of the blood being returned to the patient from the blood filtration device. In another embodiment of the invention, part of the return flow of the effectively detoxified blood is fed through the return lumen and the remainder is separately fed to the patient via a separate feed system, such as through a separate catheter feed to one of the subclavian or jugular veins as described by Krementz, supra.

The double occlusion device catheter, once properly located in the body, extends through the skin to the outside of the body. It terminates in a Luer fitting and a valve cutoff such as a stopcock. The blood filtration device can be separated from the double occlusion device catheter and reconnected at will. When the occlusion devices are not inflated/positioned, blood flow through the IVC is maintained. When the occlusion devices are inflated/positioned, the blood below the peripheral occlusion devices will find secondary pathways to the heart.

This convenience may be duplicated on the supply side of the process, where the therapeutic agent is supplied to the arterial side of the liver, via the hepatic artery, by the percutaneous insertion of a feed catheter to the hepatic artery, leaving the tubular ending of the feed catheter in a plastic reservoir surgically implanted just below the patient's skin and surgically tied therein below the skin. The plastic reservoir contains a resealing membrane of a type similar to those used in multi-dose vials that can be percutaneously penetrated from the outside of the body by one or more needles to reinitiate the flow of therapeutic agent to the diseased liver.

The double occlusion device catheter can be introduced into the femoral vein using the Seldinger technique. A guidewire made of stainless steel is first passed through a needle that has been inserted percutaneously into the vein. A catheter with a single occlusion device is inserted over the guidewire and the occlusion device is inflated/positioned to dilate the percutaneous tract to the diameter of the sheath that will transmit the double occlusion device catheter. A plastic sheath tubing is passed over the guidewire when the single occlusion device catheter is removed. After the sheath is properly located in the vein the double occlusion device catheter is inserted within the sheath and over the guidewire and advanced to the proper position relative to the organ to be treated. All manipulations of the double occlusion device catheter are done under fluoroscopic control. An inferior vena cavagram can be performed prior to catheter insertion or prior to occlusion device positioning/inflation with the patient lying on an opaque ruler, parallel to the IVC. The hepatic veins and renal veins can be identified and their location determined according to the opaque ruler.

Under fluoroscopic guidance, the catheter is positioned so that the central occlusion device, when inflated/positioned, occludes the IVC just above the hepatic veins. The peripheral occlusion device, when inflated/positioned, occludes the IVC just below the hepatic veins. Dilute contrast medium such as saline solution is used to inflate/position the occlusion devices and reference to the ruler insures accurate positioning.

In an embodiment of the invention, the double-occlusion device catheter contains three lumina. One lumen transmits an angiographic guidewire and is used for percutaneous insertion. A main lumen carries hepatic venous blood from the fenestrations between the occlusion devices to the blood filtration device. The third lumen terminates at the fenestrations and is used to measure pressure or inject contrast medium. A pressure monitor, attached to this lumen, measures pressure within the isolated segment of the vena cava before and during occlusion device inflation. The pressure measured before occlusion device inflation/positioning is the systemic venous pressure. The pressure measured after occlusion device inflation/positioning but before opening the blood filtration device is equal to the wedge hepatic venous pressure, which is assumed to be equal to portal pressure. This measurement can determine the presence or absence of portal hypertension. The pressure measured after occlusion device inflation/positioning and during flow through the blood filtration device is the hepatic venous pressure. The hepatic venous pressure can be monitored continuously during drug infusion. If a pump is used, the speed of the pump can be adjusted to maintain hepatic venous pressure above systemic venous pressure but below portal pressure. This prevents hepatic sinusoidal congestion. The calibers of the occlusion device catheter and of the tubing in the blood filtration device are calculated to ensure that they are of sufficient size to transmit the necessary volumes of blood with minimal resistance.

After inflation/positioning of the occlusion devices, an inferior vena cavagram (contrast medium injected into the inferior vena cava) is typically performed through the double occlusion device catheter prior to infusion to document complete obstruction of the vena cava proximal and distal to the hepatic veins and to demonstrate the anatomy of the hepatic veins. Samples of hepatic venous blood are generally aspirated through the pressure port of the double occlusion device catheter immediately after the beginning of infusion, and, in the typical case, at intervals not to exceed one hour during infusion, and for at least three hours after infusion, the samples are analyzed for therapeutic agent concentrations. Simultaneous blood samples are taken from the blood filtration device after detoxification and analyzed for drug concentrations in order to document the efficiency of the detoxification device in removing the drug from the blood before returning the blood to the systemic circulation. In addition, blood samples are obtained from a peripheral vein to evaluate drug concentrations reaching the systemic circulation. Systemic drug concentrations are then measured over 24 to 48 hours following the infusion.

Another double occlusion device catheter design may utilize only 2 supplemental lumina and one main lumen for blood transfer to the blood filtration device. Each supplemental lumen can supply fluids to one of the occlusion devices.

The venous pressure may provide the pressure for passage of blood to the blood filtration device. In an embodiment, a pump may be used in order to continue the movement of blood though the blood filtration device and return it to the patient. The blood is removed from the body by a combination of gravitational displacement and the venous blood pressure. The pump does not generate a negative pressure and pull blood from the body. The pressure of the return flow of the blood from the blood filtration device to the systemic venous system should be less than about 300 mm Hg.

A variety of suitable pumps are commercially available. They come in a number of designs. A preferred design is a centrifugal cardiopulmonary bypass pump that utilizes smooth surface rotators without relying on rotating vanes. These pumps have been used in long term support of cardiac bypass and in liver transplants. Such designs are shown in U.S. Pat. No. 3,487,784; Reissue 28,742; U.S. Pat. No. 3,647,324; U.S. Pat. No. 3,864,055; U.S. Pat. No. 3,957,389; U.S. Pat. No. 3,970,408 and U.S. Pat. No. 4,037,984.

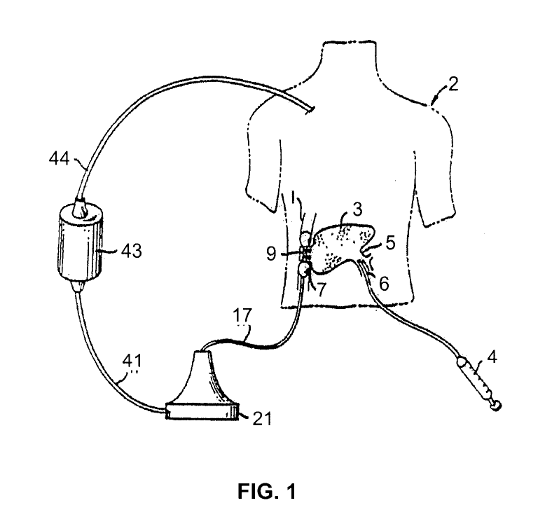

With respect to FIG. 1, there is shown the main components of a system for the in situ treatment of liver disease using local deliver of therapeutic agents, with relation to a human body 2. A liver 3 is supplied with therapeutic agents from a syringe 4 through tubing leading to a catheter 6 located in a hepatic artery 5. Hepatic venous blood containing concentrations of therapeutic agent (i.e., contaminated blood) is passed via hepatic veins to a double occlusion device catheter 9 located in the inferior vena cava (IVC). The occlusion devices of the double occlusion device catheter 9 are positioned central and peripheral of the hepatic veins. The contaminated blood is passed through the double occlusion device catheter 9 to tubing 17 to a point exterior to the body 2, then optionally to a pump 21. The pump 21 moves the contaminated blood through an extracorporeal circuit at relatively constant low pressure, the object being to avoid raising or lowering the fluid pressure of the total circuit ranging from the hepatic veins through the return to the body. The contaminated blood is transported through tubing 41 into a blood purification device 43, which will be described in more detail below, to detoxify the blood. The detoxified blood is passed through tube 44 to effect infusion through the subclavian vein (not shown) by standard procedures in the art.

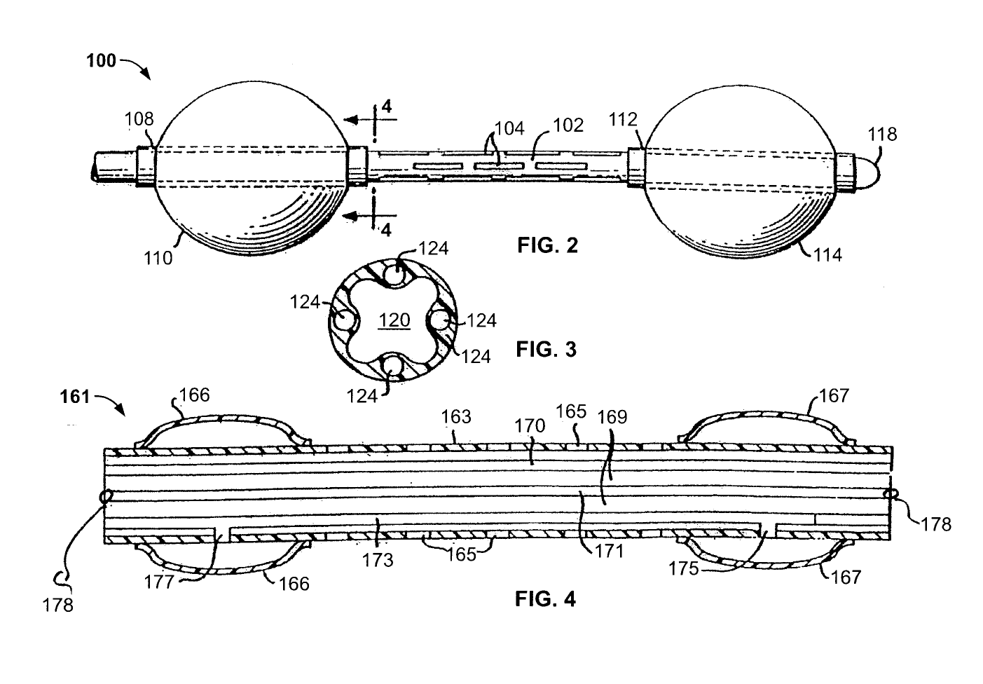

With respect to FIG. 2, there is shown an embodiment of a double occlusion device catheter 100 of the present invention. Catheter 100 includes slotted fenestrations 104 in a solid plastic tubing 102. An open end 118 terminates the catheter 100. Open end 118 is tapered to the caliber of an angiographic guide wire that will, under fluoroscope control, allow the catheter 100 to be advanced from the femoral vein to the proper location in the inferior vena cava without risk of injury to the interior of the vessels. Appropriate guide wires may be, for example, 0.035, 0.038, or 0.045 inch in diameter. During treatment, the catheter end hole is closed using a standard angiographic apparatus (tip occluding wire), that consists of a thin wire long enough to traverse the length of the catheter at the end of which is a stainless steel bead just large enough to obstruct the catheter's end-hole when advanced into it (similar to a metal stopper that closes the outlet from a sink when advanced).

Alternatively, the end hole 118 may accommodate a return catheter. The return catheter can be used to return treated blood to the systemic circulation. The return catheter is advanced over a guide wire through the main lumen of the double occlusion device catheter 100 and through the end hole 118 into the right atrium or superior vena cava. The return catheter can be made to gradually taper its O.D. by decreasing its wall thickness, leaving the I.D. constant, since the location of the tip of the return catheter is not critical. The length over which the catheter tapers is arbitrary. The taper is constructed so that the tip of the catheter is its narrowest O.D. and the O.D. increases toward the femoral vein. As this return catheter is advanced through the lumen of the main catheter 100 the tip easily passes through the end hole 118 of the double occlusion device catheter 100. The tapered end of the return catheter is advanced until it obstructs the end hole 118, preventing systemic blood from entering the double occlusion device catheter 100 when the occlusion device are inflated/positioned but leaving an open lumen through the return catheter to return blood beyond the isolated venous segment without mixing with contaminated blood.

The catheter tubing 102 can be made of a variety of plastic materials such as polypropylene, polyethylene, polyvinylchloride, ethylene vinylacetate copolymers, polytetrafluoroethylene, polyurethane, and the like. Favorable plastic combinations for catheters containing a return lumen are a homogeneous mixture of high-density polyethylene and linear low-density polyethylene. That combination gives favorable stiffness at ambient conditions and allows the use of especially thin wall thicknesses. When the surface of the catheter is made of a plastic that is difficult to bond with a occlusion device, the plastic may be treated first by one or more of a number of well known methods that make bonding possible. The methods include plasma treatment, ozone treatment, and the like. Occlusion devices 110 and 114 may be made from a plurality of materials. The occlusion devices may be adhesively bonded at sheath surfaces 108 and 112, respectively. A wide variety of adhesives may be employed. Polyacrylonitrile type adhesives, rubber latex adhesives and the like may be used to secure the occlusion device to the sheath surfaces 108 and 112.

With respect to FIG. 3, there is shown a cross section of the catheter 100 shown in FIG. 2. The interior of the catheter 100 contains a main lumen 120 and 4 additional lumina 124 molded into an outer wall. The additional lumina 124 can be used for the various functions described above.

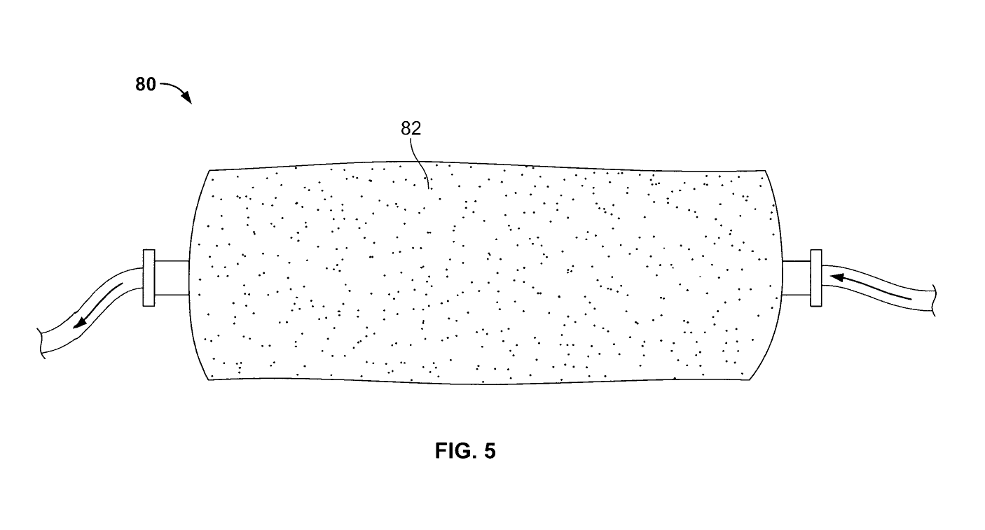

FIG. 4 provides a more detailed schematic cross sectional side view of an embodiment of a double occlusion device catheter 161. In this depiction, a catheter sidewall 163 is penetrated by a plurality of fenestrations 165. A main lumen 169 contains at its periphery supplemental lumina 170, 171 and 173. Supplemental lumens such as depicted at 170 and 171 may be closed at their distal end or may completely traverse the double occlusion device section of the catheter, with a distal opening distal to the occlusion device 167. These supplemental lumina may be utilized for any of a variety of functions. For example and without limitation, supplemental lumina 170 and 171 can be used to accommodate a guidewire and/or pressure monitor. Lumen 173 may optionally be utilized so as to inflate/position occlusion means 166 and 167.

Supplemental lumina 170 and 171 may also be utilized as a “shunt”, or bypass, as follows. A port or opening 178 may be provided in the wall of the catheter, just upstream (in the direction of blood flow) from the occlusion device 166. The port 178 may connect to a lumen 171 that extends towards the tip of the catheter, the second end of the lumen 171 positioned in the wall or the tip of the catheter just downstream or anterior to the occlusion device 167, and the second end of the lumen 171 may be provided with a second port. The two ports and the connecting lumen 171 form a shunt or bypass for the blood flowing through the vessel and blocked by the inflated/positioned occlusion devices 166 and 167. With a blood bypass, such as shown and described as part of the occlusion device catheter, isolation of the body part from the blood supply in the vessel is achieved without interfering with the flow of blood through the blood vessel.

By providing a device for uninterrupted flow of blood from upstream relative to the direction of blood flow from the obstructed segment of blood vessel to downstream relative to the direction of blood flow from the obstructed segment, the bypass mitigates the buildup of excess blood volume in the occluded blood vessel upstream of the occlusion. As blood vessels are pressure sensitive and maintain appropriate tone and resultant blood pressure at least in part through signals generated by vessel wall baroreceptors, mitigating the amount of excess blood pooling upstream of the occlusion through use of such a bypass device provides a method of minimizing the effect on baroreceptor signaling of excess, pooled blood upstream of the occluded vessel segment; and/or provides a method whereby blood rich in certain vasoactive substances (for example, renin and/or catecholamines) may be rapidly re-routed around the occluded section of the inferior vena cava or other occluded blood vessel

Remaining lumina, such as those in communication with openings 175 and 177, may be utilized to supply air and/or fluid to occlusion devices 166 and 167.

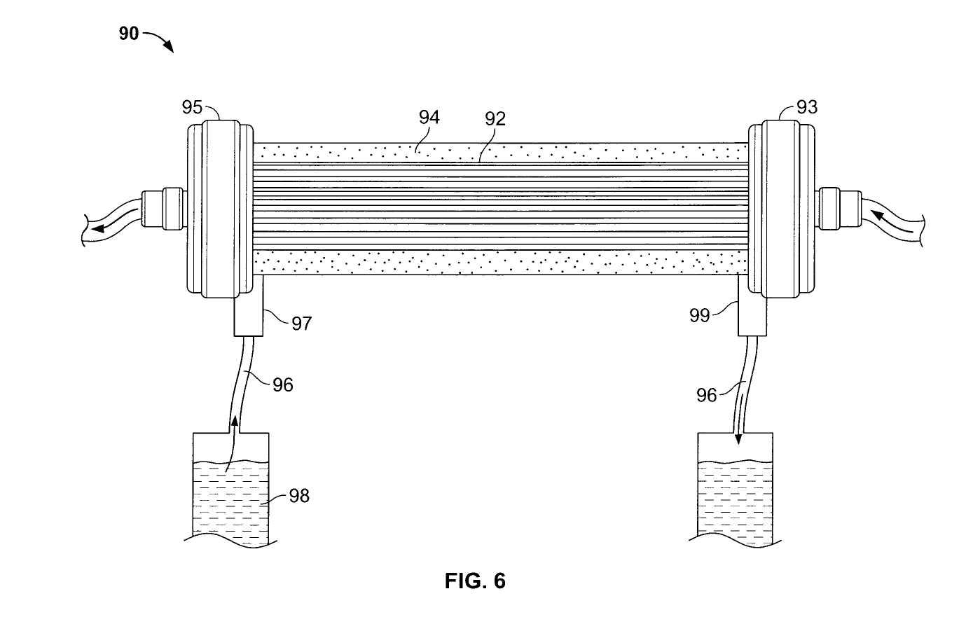

In one embodiment, the system and method of the present invention relies on the double occlusion device catheter for substantially preventing contaminated blood from entering the general circulation, as well as the blood purification device for the detoxification (treatment) of the contaminated blood. In an embodiment, the blood purification device is a cartridge, of any shape, consisting of a plastic or other material secured with two ends with ports allowing for catheter attachment. The blood purification device can further include additional ports. FIG. 5 shows a side cross-sectional view of a general cartridge-type blood purification device 80 for use with the system of the present invention. The blood purification device 80 is composed of an aggregate of blood-compatible adsorbent material 82, composed of natural, synthetic or chemical materials and which may optionally possess natural or artificially enhanced adsorbent characteristics. The blood-compatible adsorbent material 82 may be made further compatible by way of chemical, synthetic or other method of modification or coating of the adsorbent material 82 while minimally affecting adsorbent's 82 affinity characteristics. The combination of surface coating and adsorbent 82 creates a more effective filter which is less harmful to the blood and may provide additional benefits. In an embodiment, the blood purification device 80 is used to remove the chemotherapeutic agent Melphalan from contaminated blood. The blood purification device 80 may remove at least 1.5 mg/kg Melphalan from human blood and is capable of flow rates exceeding about 500 ml/minute/device. In other embodiments, the flow rates can vary. Drug removal will ideally begin at 90-100% removal rates, and gradually decrease in efficiency as the infusion progresses. In other embodiments, the efficacy will remain constant throughout the detoxification process. Total efficiency for drug removal will be between about fifty and about one-hundred percent of drug delivered.

As shown in the embodiment depicted in FIG. 6, a blood purification device 90 is a hollow-fiber device. The blood purification device 90 comprises a canister cartridge consisting of, within a hollow portion of the cartridge, hollow fibers 92 connected to each end (93 and 95) of the cartridge, whereby treated blood flows through the hollow fibers 92 in one direction, from end to end, and whereby the hollow fibers 92 within the cartridge are surrounded by a natural, synthetic or chemical based adsorbent material 94 which assists in the adsorption of the agents from the treated blood. The cartridge possesses a flow through capability external to the hollow fibers 92 and internal of the cartridge, to continually flush the adsorbent material 94 of the blood purification device 90, allowing for increased adsorption capability by way of preventing saturation. The flow through capability is achieved by two ports (97 and 99) on each end of the cartridge attached to tubes 96 for the continuous one way flow of a flushing agent 98. In an embodiment, flow direction of the treated blood, hollow fibers 92, and flushing agent 98 can vary. The hollow fibers 92 are composed of a porous material, allowing for the pass through of the therapeutic agent for the adsorption by surrounding adsorbent material 94. Membrane permeability is assisted by way of negative pressure, fluctuating pressure or other device of pressure gradient or circular flow. In an embodiment, the membrane permeability, method of permeability and composition can vary. The adsorbent material 94 is composed of natural, synthetic or chemical materials and may possess natural or artificially enhanced adsorbent characteristics. In an embodiment, the blood purification device 90 is used to remove the chemotherapeutic agent Melphalan from contaminated blood. The blood purification device 90 may remove at least 1.5 mg/kg Melphalan from the human blood and is capable of flow rates exceeding about 500 ml/minute/device for a period of time not less than about one minute and not more than about four hours. In additional embodiments, the flow rate and absorption efficacy can vary. Devices 90 are capable of being run simultaneously in parallel, and singly, whereby a single device 90 will possess the capacity to handle the adsorption, pressure, and other requirements of it, alone. At least two devices 90 are capable of being run laterally to each other, end-to-end in series, with an adaptor connecting the two devices 90, whereby the adaptor is designed specifically for connecting the devices 90. A variety of adsorbent slurries can be used in this device 90 depending on which therapeutic agent is trying to be extracted. Different slurries can be used in each device 90 if run laterally. Reconstitution could occur in the second device 90 if run laterally. The effect of using hollow fibers 92 through the device 90 with the adsorbent material 94 surrounding the hollow fibers 92, creates a synergistic and maximized filtration of therapeutic drug agent.

Suitable adsorbent materials for use with any of the blood purification devices of the present invention include, but are not limited to, carbon-based adsorbent materials, coated or uncoated with a biocompatible synthetic, natural or chemical coating or modification, geared to minimize impact on the blood while minimally affecting the adsorbent characteristics of the carbon-based adsorbent. Such coating may include, for example, methyl methacrylate. Adsorbents may be prepared by coating crushed carbon originated from vegetables (hereinafter referred to as the coated crushed active carbon), for example, or an active carbon made of carbonized shell of coconut (hereinafter referred to as the coated coconut active carbon). Coated crushed active carbon may be prepared, for example, by dipping the original carbon into an ethyl alcohol-ethyl ether solution of pyroxylin, and drying the same. Coated coconut active carbon may be prepared, for example, by coating the original carbon into an ethyl alcohol-ethyl ether solution of pyroxylin via a phase separation process using dioxan as a solvent.

The blood purification device may use a coated bead-shaped activated carbon for the purification of the blood, which is prepared by coating a beads-shaped activated carbon with a film-forming material. The bead-shaped activated carbon that may be used in the blood purification device of the present invention is an active carbon having a nearly perfect sphere form, which is obtained from pitch as a source material through melt molding, that is, a process for the molding of melted material. The bead-shaped activated carbon is different from conventional crushed or granulated active carbon. More particularly, the bead-shaped activated carbon can be prepared by, for example, dispersing the pitch in melted state into water to form a sphere, making the sphere non-fusible and carbonizing the same. As for detailed descriptions of the preparation for the bead-shaped activated carbon, refer to Japanese Patent Publication Nos. 25117/74 and 18879/75, for example. Such bead-shaped activated carbon is available in the market under the name of bead-shaped activated carbon (BAC) [Trade Mark, manufactured and sold by Taiyokaken Kabushiki Kaisha in Japan]. The film-forming material is selected from the materials which may provide a semipermeable film, including, but not limited to, pyroxylin, polypropylene, copolymer of vinyl chloride-vinylidene chloride, ethylene glycol polymethacrylate, collagen, and the like, for example. A conventional process may be employed for coating the beads-shaped activated carbon with the film-forming materials. Examples of such processes include, but are not limited to, pan coating, air suspension coating, spray drying, and the like. As a solvent to be employed for dissolving the film-forming material in the coating process, it is desirable to use a solvent which can be easily removed at a drying step, and has a low toxicity even if the solvent is dissolved into the blood. In view of this point, ethanol is an especially preferred solvent, when pyroxylin is used for the film-forming material.

When the coated bead-shaped activated carbon is used for the purification of the blood, it may be desirable to further coat the coated bead-shaped activated carbon. The activated carbon may be further coated with methyl methacrylate or albumin. In additional embodiments, varying adsorption compositions may be used other than carbon-based.

Any of the blood purification devices of the present invention may utilize, in addition to the carbon-based binding characteristics, biotin-avidin, antibody-antigen, and/or other protein affinity interactions. These interactions rely on the process of tagging the therapeutic agent with the biotin and tagging the adsorbent material of the blood purification device with opposite attracting agent, avidin, whereby the binding of the avidin to the carbon has minimal negative impact on the adsorbent characteristics. In other embodiments, varying affect on the binding and affinity and adsorption characteristics may be had. This method of blood filtration relies on both carbon adsorption and biotin-avidin attraction. In additional embodiments, interactions based on affinity relationships other than biotin-avidin may be utilized. The effect of combining the protein-based affinity characteristics with the preexisting adsorbent characteristics and the double occlusion deviceocclusion device catheters of the invention creates a highly effective and maximized method of drug filtration from blood. Such filtration technologies may also optionally be applied to the removal of certain agents, for example, vasoactive or other biologically active agents, from the blood.

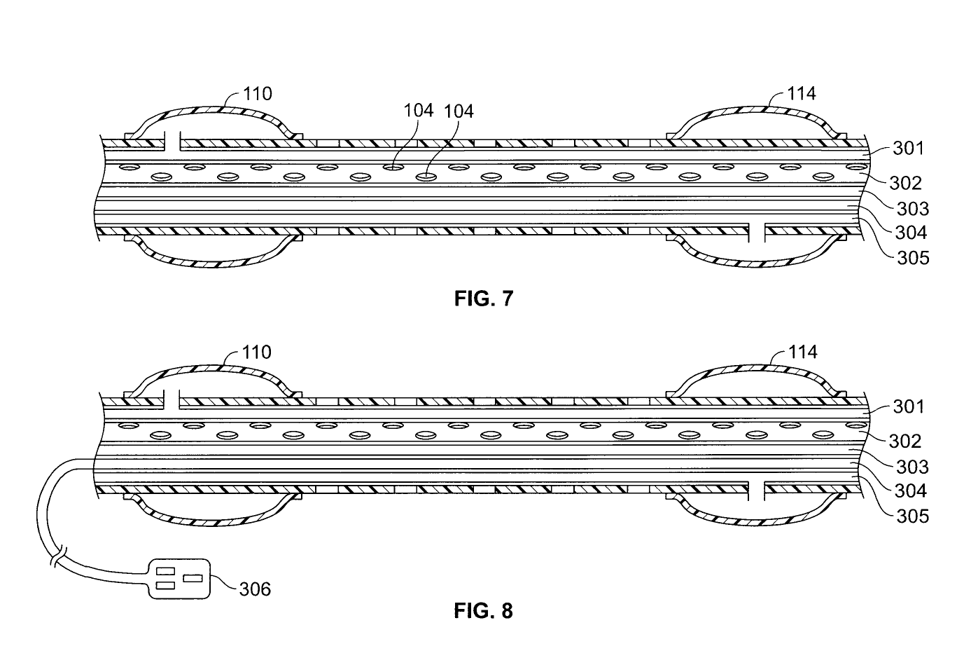

FIGS. 7-9 provide more detail as to a second, alternative and/or complementary device of providing for bypass of uncontaminated blood from upstream to downstream of the occluded vessel section; and/or for controlling blood pressure disturbances that may be occasioned by such occlusion.

FIG. 7 discloses a five-lumen catheter device. First lumen 301 may be utilized to inflate/position a first occlusion device 110. Second lumen 302 has perforations 104 that may be utilized to convey blood draining into the occluded space between the two inflated/positioned occluding devices 110, 114 to an extracorporeal variable speed pump and filtering device (not shown). Third lumen 303 is a bypass lumen, open at both ends, that may shunt the occluded section of a blood vessel by providing a passage for blood to flow through from upstream to downstream of the occluded segment of the blood vessel. Fourth lumen 304 is a bypass lumen that is connectable to an extracorporeal circuit for uncontaminated blood, as described in further detail below, and that is optionally dimensioned and sized so as to accommodate an adequate blood flow rate for upstream blood draining into the inferior vena cava that must be bypassed around the occluded section of the inferior vena cava. Fifthlumen 305 may be utilized to inflate/position a second occlusion device 114.

FIG. 8 discloses a five lumen catheter device. First lumen 301 may be utilized to inflate/position a first occlusion device 110. Second lumen 302 has perforations that may be utilized to convey blood draining into the occluded space between the two inflated/positioned occluding device 110, 114 to an extracorporeal variable speed pump and filtering device (not shown). Third lumen 303 is a bypass lumen, open at both ends, that may shunt the occluded section of a blood vessel by providing a passage for blood to flow through from upstream to downstream of the occluded segment of the blood vessel. Fourth lumen 304 is a bypass lumen that is connected to an extracorporeal circuit for uncontaminated blood having a device for collecting upstream blood 306, as described in further detail herein. Fifth lumen 305 may be utilized to inflate/position a second occlusion device 114.

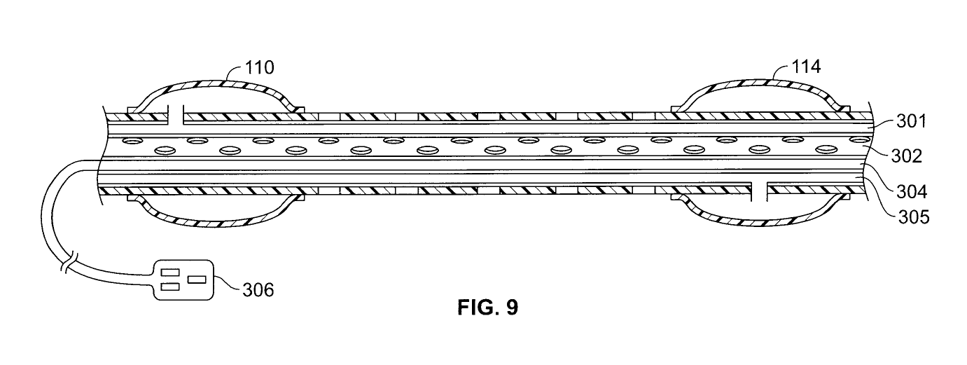

FIG. 9 discloses a four-lumen catheter device. First lumen 301 may be utilized to inflate/position a first occlusion device 110. Second lumen 302 has perforations that may be utilized to convey blood draining into the occluded space between the two inflated/positioned occluding device 110, 114 to an extracorporeal variable speed pump and filtering device (not shown). Third lumen 304 is a bypass lumen that is connected to an extracorporeal circuit for uncontaminated blood having a device for collecting upstream blood 306, as described in further detail below. Fourth lumen 305 may be utilized to inflate/position a second occlusion device 114.

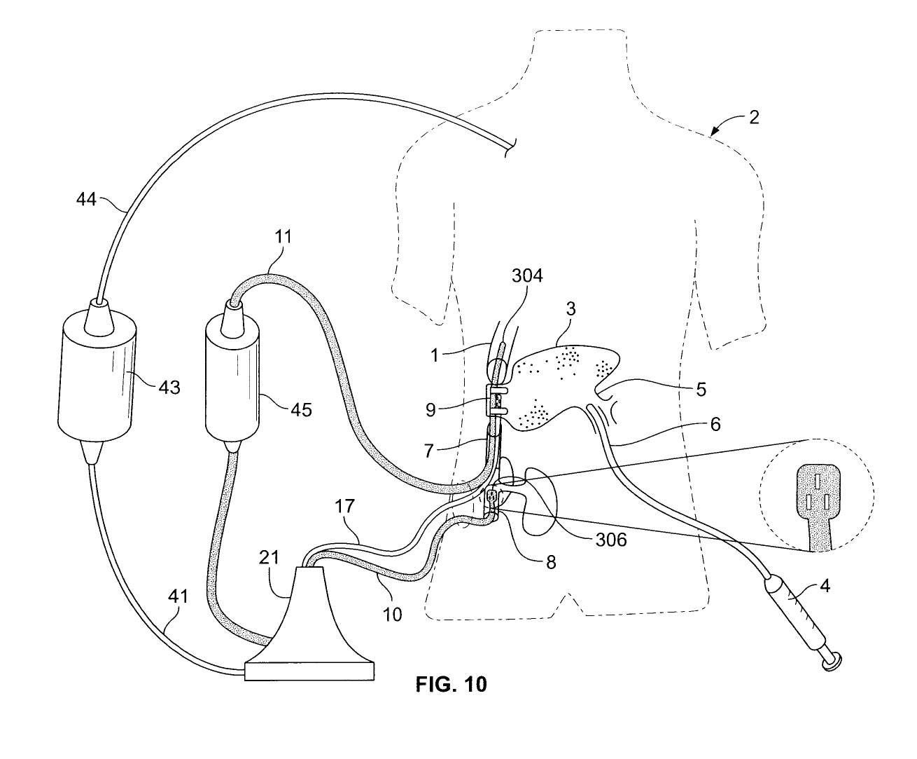

FIG. 10 depicts one embodiment of the instant invention in operation. A liver 3 is supplied with therapeutic agents from a syringe 4 through tubing leading to a catheter 6 located in a hepatic artery 5. Hepatic venous blood containing concentrations of therapeutic agent (i.e., contaminated blood) is passed via hepatic veins to a double occlusion device catheter 9 located in inferior vena cava (IVC) 1. The occlusion device of the double occlusion device catheter 9 are positioned central and peripheral of the hepatic veins. The contaminated blood is passed through the double occlusion device catheter 9 to tubing 17 to a point exterior to the body 2, then optionally to a pump 21. The pump 21 optionally moves the contaminated blood through an extracorporeal circuit at relatively constant low pressure, the object being to avoid raising or lowering the fluid pressure of the total circuit ranging from the hepatic veins through the return to the body. The contaminated blood is transported through tubing 41 and optionally flows through a blood purification device 43, which will be described in more detail below, to detoxify the blood. The detoxified blood is passed through tube 44 to effect infusion through the subclavian vein (not shown) by standard procedures in the art.

Renal venous blood is passed via renal veins to a catheter collection device 8 located in or proximal to a renal vein, upstream of the double occlusion catheter. The collection device of an excess upstream blood collection catheter 10 is positioned proximal to at least one renal vein. This collected renal blood is passed to a point exterior to the body 2, then optionally to a pump 21. The pump 21, which may be the same pump as that used for the contaminated blood circuit or a separate unit (not shown), moves the renal venous blood through an extracorporeal circuit at relatively constant low pressure, the object being to avoid raising or lowering the fluid pressure of the total circuit ranging from the renal veins through the return to the body. The pump 21 is optionally designed so as to accommodate an adequate blood flow rate for upstream blood draining into the inferior vena cava that must be bypassed around the occluded section of the inferior vena cava. The renal venous blood is transported through tubing 42, optionally into a blood purification device 45, which will be described in more detail below, which may optionally remove compounds of interest (in one embodiment, renin, catecholamines and/or other vasoactive substances or constituents of the renin-angiotensin-aldosterone axis) from the renal venous blood. The filtered renal venous blood is passed through tube 11 and is returned to the patient downstream of the occluded blood vessel segment via return lumen 304, which lumen may optionally extend proximate to the corresponding atrium.

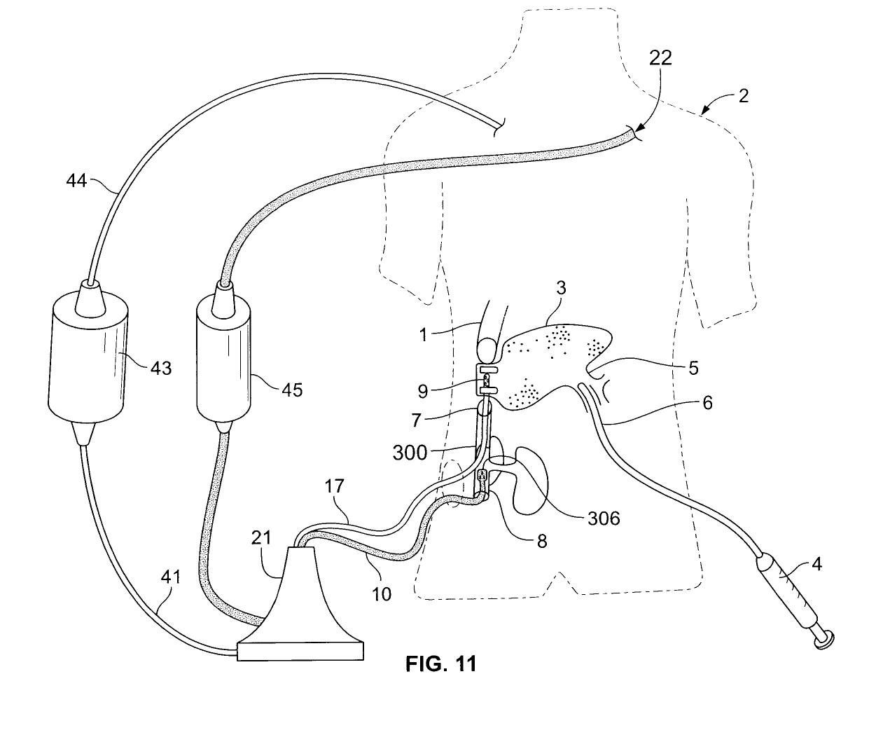

FIG. 11 depicts an alternate embodiment of the instant invention in operation. A liver 3 is supplied with therapeutic agents from a syringe 4 through tubing leading to a catheter 6 located in a hepatic artery 5. Hepatic venous blood containing concentrations of therapeutic agent (i.e., contaminated blood) is passed via hepatic veins to a double occlusion device catheter 9 located in inferior vena cava (IVC) 1. The occlusion device of the double occlusion device catheter 9 are positioned central and peripheral of the hepatic veins. The contaminated blood is passed through the double occlusion device catheter 9 to tubing 17 to a point exterior to the body 2, then optionally to a pump 21. The pump 21 moves the contaminated blood through an extracorporeal circuit at relatively constant low pressure, the object being to avoid raising or lowering the fluid pressure of the total circuit ranging from the hepatic veins through the return to the body. The pump 21 is optionally designed so as to accommodate an adequate blood flow rate for upstream blood draining into the inferior vena cava that must be bypassed around the occluded section of the inferior vena cava. The contaminated blood is transported through tubing 41 into a blood purification device 43, which will be described in more detail below, to detoxify the blood. The detoxified blood is passed through tube 44 to effect infusion through the subclavian vein (not shown) by standard procedures in the art.

Renal venous blood is passed via renal veins to a catheter collection device 8 located in a renal vein, upstream of the double occlusion catheter. The collection device of an excess upstream blood collection catheter 10 is positioned proximal to at least one renal vein. The collected renal blood is passed to a point exterior to the body 2, then optionally to a pump 21. The pump 21, which may be the same pump as that used for the contaminated blood circuit or a separate unit (not shown), moves the renal venous blood through an extracorporeal circuit at relatively constant low pressure, the object being to avoid raising or lowering the fluid pressure of the total circuit ranging from the renal veins through the return to the body. The renal venous blood is transported through tubing 42, optionally into a blood purification device 45, which will be described in more detail below, which may optionally remove compounds of interest (in one embodiment, renin and other angiotensive hormones) from the renal venous blood. The filtered renal venous blood is passed through tube 11 and is returned to the patient to effect infusion 22 through a remote blood vessel (not shown) by standard procedures in the art.

The extracorporeal bypass loop consists of the following components:
device (304) for returning uncontaminated blood to the subject. Of note, such a return may be directly to the downstream side of downstream occlusion device 114 or may be to a remote location, e.g. a jugular vein or proximal to the atrium (akin to the return of treated blood shown at FIG. 11, 22). The device 304 is optionally designed so as to accommodate an adequate blood flow rate for upstream blood draining into the inferior vena cava that must be bypassed around the occluded section of the inferior vena cava.
optional pump (21) for pumping renal venous blood from its removal point to its return point. Of note, such a pump may be a second, standalone pump or may be a dual-purpose pump that both circulates renal venous blood in the uncontaminated extracorporeal circuit and circulates contaminated blood in a second, extracorporeal circuit. The pump 21 is optionally designed so as to accommodate an adequate blood flow rate for upstream blood draining into the inferior vena cava that must be bypassed around the occluded section of the inferior vena cava.
optional filtering device (45) for filtering uncontaminated blood, in line with the renal venous extracorporeal bypass circuit.
device 306 for withdrawal of uncontaminated blood from a location upstream from occlusion device 110 in FIG. 4.

In operation, withdrawal device 306 is placed in a location of a catheter-occluded blood vessel upstream of the occlusion. The withdrawal device 306 may optionally be placed by insertion in a femoral vein or artery not utilized in placement of the double occlusion device catheter. Uncontaminated blood flows into the withdrawal device 306, is optionally pumped 21 through an extracorporeal circuit and is reintroduced to the subject either through a lumen opening downstream of occlusion device 304 or at a site remote from the occlusion, e.g. as shown and described in FIG. 11, 22.

In another embodiment, withdrawal device 306 is sized and dimensioned so as to be specifically placed proximate to or within the renal vein.

Renin is a peptide hormone that is secreted by the kidney from specialized cells called granular cells of the juxtaglomerular apparatus in response to:
A decrease in arterial blood pressure (that could be related to a decrease in blood volume) as detected by baroreceptors (pressure sensitive cells). This is the most causal link between blood pressure and renin secretion (the other two methods operate via longer pathways).
A decrease in sodium chloride levels in the ultra-filtrate of the nephron. This flow is measured by the macula densa of the juxtaglomerular apparatus.
Sympathetic nervous system activity, that also controls blood pressure, acting through the β₁ adrenergic receptors.

Renin acts to hydrolyze angiotensin, resulting in increased plasma levels of angiotensin 1 and a downstream result of increased blood pressure occasioned by vasoconstriction.

Catecholamines are sympathomimetic “fight-or-flight” hormones that are released by the adrenal glands in response to stress. They are part of the sympathetic nervous system, are found in the renal vein microenvironment, and can cause increased heart rate and increased blood pressure.

Renin, catecholamine, and/or other vasoactive substance levels may be increased as a physiological response to occlusion of a blood vessel and/or extracorporeal filtration of contaminated blood, both of which may be accompanied by a drop in blood pressure. Withdrawal device 306 may optionally be placed by insertion in a femoral vein or artery not utilized in placement of the double occlusion device catheter. Uncontaminated blood from the renal vein that may have elevated levels of renin, catecholamines and/or other vasoactive substances flows into the withdrawal device 306, and may be pumped 21 through an extracorporeal circuit. Optional in-line filter device 45 may be utilized to filter out components of the plasma found in the renal vein microenvironment, utilizing techniques described elsewhere in the instant application; or, alternately, the extracorporeal circuit may function to ensure that plasma levels of renin and/or catecholamines found in the renal vein microenvironment are more quickly delivered to the remaining systemic circulation by providing a high-throughput bypass around the occluded segment of the inferior vena cava. The uncontaminated, optionally filtered blood is reintroduced to the subject either through a lumen opening downstream of occlusion device 304 or at a site remote from the occlusion, e.g. as shown and described in FIG. 11, 22. Rapid return of renal vein microenvironment renin and/or catecholamines to the systemic circulation may result in an increased systemic circulation plasma level of these hormones and result in an improved ability of the subject to maintain blood pressure homeostasis by responding to a drop in blood pressure occasioned by occlusion of the inferior vena cava and/or filtration of blood in an extracorporeal circuit.

Though this invention has been described with emphasis on the treatment of liver disease resulting from cancer and viruses, it is quite apparent that the invention has broader application. The invention is useful for the treatment of any organ in which the treating agent would cause toxological effects if it entered the body's general circulation. For example, the invention could be applied to the treatment of infectious diseases of organs such as fungal diseases. A specific illustration would be the treatment of hepatic fungal infections with Amphotericen B. The procedures described above would be directly applicable to extracorporeal recovery of this agent and its isolation from entering the general circulation of the body during treatment of the liver with significant concentrations of this drug.

Therefore, the breadth of the invention encompasses the perfusing of a high concentration of an agent to treat an organ, such as anti-cancer agents through a body organ containing a tumor, without their entering the body's general circulation, removing them from the organ with effluent blood and transporting the contaminated blood to an extracorporeal blood purification device where the blood is treated to remove the contamination, and returning the treated blood to the body. The process prevents toxic levels of the agents from entering the body's general circulation while delivering lethal doses of the agents to the tumor. While illustrative embodiments of the invention are disclosed herein, it will be appreciated that numerous modifications and other embodiments may be devised by those skilled in the art. Therefore, it will be understood that the appended claims are intended to cover all such modifications and embodiments that come within the spirit and scope of the present invention.

1-7. (canceled)

8. A system for isolating a liver undergoing treatment with a chemotherapeutic agent and maintaining blood flow past the isolated liver in a patient, comprising:
a double occlusion catheter suitable for placement in the inferior vena cava and capable of isolating hepatic blood contaminated with the chemotherapeutic agent from the liver by forming an occluded region of the inferior vena cava, and
a blood bypass from a position upstream of the occluded region capable of substantially maintaining normal flow of uncontaminated blood occluded through the inferior vena cava.

9. The system of claim 8, wherein the double occlusion catheter comprises a first occlusion device and a second occlusion device oriented in the direction of blood flow in the inferior vena cava from the first occlusion device to the second occlusion device, the first occlusion device and the second occlusion device expandable beyond a wall of the double occlusion catheter and spaced along the catheter for generating an occluded segment of the inferior vena cava when the first occlusion device and the second occlusion device are expanded, and a fenestrated lumen for collection of hepatic blood contaminated with the chemotherapeutic agent.

10. The system of claim 9, wherein the first occlusion device and the second occlusion device are balloons.

11. The system of claim 8, wherein the blood bypass is from a position of the double occlusion catheter upstream of the occluded region to a position downstream of the occluded region.

12. The system of claim 11, wherein the blood bypass comprises:
a first port in the wall of the double occlusion catheter, the first port positioned upstream, in the direction of the blood flow from the first occlusion device;
a second port positioned downstream, in the direction of the blood flow from the second occlusion device, and
a lumen within the double occlusion catheter and having a first end and a second end, the first end connected to the first port and the second end connected to the second port for defining a bypass for blood to maintain substantially normal blood flow by shunting the occluded region of the inferior vena cava.

13. The system of claim 8, wherein the blood bypass is an extracorporeal circuit from a position upstream of the double occlusion catheter to a position downstream of the double occlusion catheter.

14. The system of claim 13, wherein the position upstream of the double occlusion catheter is in a renal vein.

15. The system of claim 13, wherein the position upstream of the double occlusion catheter is proximal to a renal venal.

16. The system of claim 13, wherein the extracorporeal circuit comprises a catheter collection device located in or proximal to a renal vein upstream of the double occlusion catheter.

17. The system of claim 13, wherein the extracorporeal circuit comprises a device for returning uncontaminated blood to a position downstream of the double occlusion catheter.

18. The system of claim 17, wherein the position downstream of the double occlusion catheter for return of the uncontaminated blood is the jugular vein.

19. The system of claim 17, wherein the position downstream of the double occlusion catheter for return of the uncontaminated blood is proximal to the atrium.

20. The system of claim 13, wherein the extracorporeal circuit comprises a pump for moving blood through the extracorporeal circuit.

21. The system of claim 8, further comprising a delivery catheter for arterial delivery of the chemotherapeutic agent to the liver.

22. The system of claim 8, wherein vasoactive substance levels are maintained in the patient.

23. The system of claim 22, wherein the vasoactive substance is a catecholamine.

24. The system of claim 22, wherein the vasoactive substance is renin.

25. A system for isolating a liver undergoing treatment with a chemotherapeutic agent by forming an occluded region of the inferior vena cava to isolate hepatic blood flow from the liver and maintaining blood flow by bypassing the occluded region, comprising:
a double occlusion catheter suitable for placement in the inferior vena cava and capable of isolating hepatic blood flow from the liver, the double occlusion catheter comprising
a first occlusion device and a second occlusion device oriented in the direction of blood flow in the inferior vena cava from the first occlusion device to the second occlusion device, the first occlusion device and the second occlusion device expandable beyond a wall of the double occlusion catheter and spaced along the catheter for generating an occluded segment of the inferior vena cava when the first occlusion device and the second occlusion device are expanded, and a fenestrated lumen for collection blood from the liver contaminated with the chemotherapeutic agent;
means for removing uncontaminated blood from a location upstream of the first expandable occlusion device; and
means for reintroducing the uncontaminated blood into a subject downstream from the second occlusion device.

26. A system for occluding a blood vessel and maintaining blood flow therethrough, comprising:
an occlusion catheter for placement in the blood vessel, and
an extracorporeal blood flow bypass.

27. A double occlusion catheter for occluding a blood vessel and providing a blood flow bypass, comprising:
a first occlusion device and a second occlusion device the first occlusion device and the second occlusion device expandable beyond a wall of the double occlusion catheter and spaced along the catheter for generating an occluded segment of the blood vessel when the first occlusion device and the second occlusion device are expanded, and
one or more bypass lumen capable of maintaining a blood flow bypass of the occluded segment of the blood vessel of greater than 500 ml/min.

28. The double occlusion catheter of claim 27, wherein there are two or more bypass lumens.

29. The double occlusion catheter of claim 27, wherein the blood vessel is the inferior vena cava.

30. A method of isolating a liver of a patient undergoing treatment with a chemotherapeutic agent directed to the liver while maintaining vasoactive substance levels, comprising:
forming an occluded region of the inferior vena cava to isolate hepatic blood from the liver contaminated with the chemotherapeutic agent of the patient undergoing treatment with a chemotherapeutic agent directed to the liver;
forming a blood bypass from a position upstream of the occluded region to collect uncontaminated renal venous blood, and
returning the uncontaminated blood to the patient to a position downstream of the occluded region.

31. The method of claim 30, wherein the blood bypass comprises a catheter collection device located in the renal vein.

32. The method of claim 31, wherein the catheter collection device located in the renal vein is connected to an extracorporeal circuit for returning the uncontaminated renal venous blood to the patient downstream of the occluded region.

33. The method of claim 30, wherein the position downstream of the occluded region is the jugular vein of the patient.

34. The method of claim 30, wherein the vasoactive substance is a catecholamine.

35. The method of claim 30, wherein the vasoactive substance is renin.

36. The method of claim 30, further comprising collecting the hepatic blood contaminated with chemotherapeutic agent, purifying the hepatic blood contaminated with chemotherapeutic agent, and returning the purified blood to the patient.
